(19) United States
(12) Patent Application Publication
(10) Pub. No.: US 2025/0248916 A1
(43) Pub. Date: Aug. 7, 2025

(54) NOVEL USE OF PSICOSE

(71) Applicant: DSM IP Assets B.V., Maastricht (NL)

(72) Inventors: Stephan DOPPLER, Kaiseraugst (CH); Lise Anne KOHLER, Kaiseraugst (CH)

(21) Appl. No.: 18/855,124

(22) PCT Filed: Apr. 13, 2023
(86) PCT No.: PCT/EP2023/059722

§ 371 Date: Oct. 8, 2024

(30) Foreign Application Priority Data
Apr. 14, 2022 (EP) 22168287.5

(51) Int. Cl. A61K 8/60; A61K 8/34; A61K 8/67; A61Q 19/00

(52) U.S. Cl. CPC A61K 8/60; A61K 8/345; A61K 8/673; A61Q 19/00

(57) ABSTRACT

The present invention relates to the use of psicose to suppress the discoloration of vitamin B12 in aqueous compositions. Furthermore, the invention relates to aqueous compositions comprising psicose, Vitamin B12 and optionally one or more preservatives selected from phenoxyethanol, (ethyl)hexylglycerin and/or an 1,2-alkandiol.

The present invention relates to the use of psicose to suppress the discoloration of vitamin B12 in aqueous compositions. Furthermore, the invention relates to aqueous compositions comprising psicose, Vitamin B12 and one or more preservatives selected from phenoxyethanol, (ethyl)hexylglycerin and/or an 1,2-alkandiol.

Vitamin B12 also known as cyanocobalamin is a popular cosmetic ingredient used to relieve sensitive and stressed skin as well as to treat itchy, irritated, inflamed, red and cracked skin. Vitamin B12 has also been reported to help to prevent photo-damaged skin and to protect skin barrier damage e.g., induced by inflammation. Furthermore, Vitamin B12 plays an important part in speeding up cell recovery and regeneration, thus making skin look more vibrant. In addition, Vitamin B12 provides a nice and pleasant pink color to cosmetic products.

Vitamin B12, however, tends to discolor upon storage in aqueous compositions, which is highly unwanted as it leads to an unpleasant optical, often brownish) appearance of the respective product.

In accordance with the present invention, it has now surprisingly been found that the discoloration of aqueous compositions containing Vitamin B12 can be effectively reduced by the addition of psicose. Said effect is particularly pronounced in aqueous compositions comprising in addition phenoxyethanol, (ethyl)hexylglycerin and/or an 1,2-alkanediol.

Thus, in a first embodiment, the present invention is concerned with the use of psicose for suppressing discoloration in aqueous compositions comprising Vitamin B12. Preferably, said aqueous composition further comprises one or more preservatives selected from the group consisting of phenoxyethanol, (ethyl)hexylglycerin and 1,2-alkandiols.

In another embodiment the invention is concerned with a method for reducing discoloration of Vitamin B12 in an aqueous composition, said method comprising preparing an aqueous composition by admixing Vitamin B12, psicose, and water to obtain a composition having a reduced tendency for discoloration caused by Vitamin B12 compared to a composition not comprising psicose. The method preferably comprises storing the respective composition for at least 1, more preferably for at least 2, most preferably for at least 4 weeks, such as e.g. for 6 weeks or 3 months, preferably at room temperature (i.e. about 22° C.) or at 50° C. Even more preferably, said aqueous composition further comprises one or more preservatives selected from the group consisting of phenoxyethanol, (ethyl)hexylglycerin and 1,2-alkandiols which are also admixed into the aqueous composition.

In a further aspect the invention relates to the use of a combination of Vitamin B12 and psicose, preferably in combination with one or more preservatives selected from the group consisting of phenoxyethanol, (ethyl)hexylglycerin and 1,2-alkandiols for the preparation of storage (i.e. color) stable compositions. The compositions exhibit an excellent storage stability in view of preventing/suppressing discoloration caused by Vitamin B12. Said compositions can be prepared by admixing Vitamin B12, psicose, preferably in combination with one or more preservatives selected from the group consisting of phenoxyethanol, (ethyl)hexylglycerin and 1,2-alkandiols and water.

The term ‘aqueous composition’ as used herein refers to compositions which comprise water.

In all embodiments of the present invention the aqueous compositions preferably do not contain rutin.

The term ‘supress/suppressing discoloration’ as used herein refers to a reduced discoloration of the compositions according to the present invention compared to a control not comprising the mixture according to the present invention. The suppression of discoloration according to the present invention can be assessed visually and/or by measuring the b-values (according to the CIELAB colorspace), whereas the b-values are reduced upon storage, such as upon storage for at least 2 weeks compared to a respective control not comprising the sugar blend as outlined in the example.

The term ‘Vitamin B12’ as used herein refers to cyanocobalamine [Cas No. 68-19-9], which is e.g. available as as Quali®-B or Vitamin B12 Cryst Food Grade at DSM Nutritional Products AG, (4303 Kaiseraugst, Switzerland).

Psicose is commercially available e.g. at Sigma Aldrich as D-psicose (CAS 551-68-8), which is particularly preferred in all embodiments herein.

Alternatively the psicose can be used in the form of a sugar blend obtainable by isomerization of glucose. Said isomerization is well known to a person skilled in the art. Preferably, the isomerization process comprises (a) dissolving glucose in water followed by (b) isomerization said glucose in the presence of a base, preferably in the presence of sodium hydroxide, more preferably at a temperature selected in the range from 25 to 100° C. and (c) purifying the resulting reaction mixture by chromatography and optionally filtration. Said sugar blends typically comprise from 1 to 5 wt.-%, preferably from 2 to 3 wt.-% of psicose. The total amount of sugar blend to be incorporated into the aqueous compositions according to the present invention can easily be adjusted by a person skilled in the art, based on the psicose content. The use of said sugar blend further improves the storage stability.

Preferably, the use level of Vitamin B12 in all embodiments of the present invention is selected in the range from 0.0001 wt.-% to 1 wt.-%, preferably from 0.0001 wt.-% to 0.5 wt.-%, preferably in the range from 0.001 wt.-% to 0.25 wt.-%, most preferably in the range from 0.001 to 0.1 wt.-%. Further suitable ranges include from 0.0025 to 0.1 wt.-%, 0.005 wt. % to 0.075 wt.-%, 0.005 to 0.05 wt.-%, 0.0075 wt. % to 0.1 wt.-%, 0.0075 wt. % to 0.075 wt.-%, as well as 0.0075 wt. % to 0.05 wt.-%, based on the total weight of the aqueous composition.

Preferably, the use level of psicose in all embodiments of the present invention is selected in the range from 0.005 to 0.5 wt.-%, more preferably in the range from 0.005 to 0.25 wt.-%, most preferably in the range from 0.01 to 0.5 wt.-% such as in the range from 0.1 to 0.5 wt.-%, based on the total weight of the composition. Further suitable ranges encompass 0.05 to 1 wt.-%; 0.05 to 0.5 wt.-%, 0.05 to 0.35 wt.-%, 0.075 to 0.5 wt.-%, 0.1 to 0.5 wt.-%, 0.1 to 0.5 wt.-%, and 0.15 to 0.35 wt.-%, based on the total weight of the aqueous composition.

Advantageously, in all embodiments of the present invention, the weight-ratio (w/w) between the psicose and the Vitamin B12 is selected in the range from 100:1 to 1:100, preferably from 50:1 to 1:50, more preferably in the range from 25:1 to 1:25, most preferably in the range from 25:1 to 1:1, such as I the range from 25:1 to 5:1.

In all embodiments of the present invention, the total amount of water in the aqueous composition according to the present invention is advantageously at least 20 wt.-%, preferably at least 30 wt. %, more preferably at least 40 wt.-%, most preferably at least 45 wt.-%, such as in particular in the range from 50 to 99 wt.-% of water, based on the total weight of the aqueous composition

Even more advantageously, in all embodiments of the present invention the water content in the aqueous compositions according to the present invention is selected in the range from 30 to 99 wt.-%, from 40 to 99 wt.-%, from 45 to 99 wt.-% or from 50 to 99 wt.-%, based on the total weight of the aqueous composition. Further suitable ranges are from 30 to 75 wt.-%, from 30 to 70 wt.-%, from 30 to 60 wt.-% and from 40 to 60 wt.-%.

In all embodiments according to the present invention including all compositions, methods and uses as disclosed herein, the aqueous compositions preferably are topical compositions, i.e. compositions intended to be applied to the skin and/or scalp.

Even more preferably, the compositions are topical cosmetic (non-therapeutic) compositions intended for beautifying the skin or the scalp i.e. used to treat, care for or improve the appearance of the skin and/or the scalp.

In all embodiments of the present invention, the compositions preferably further comprise one or more preservatives selected from the group consisting of phenoxyethanol, (ethyl)hexylglycerin and 1,2-alkandiols, preferably from phenoxyethanol, ethylhexylglycerin and/or 1,2-hexandiol. Said compositions are still novel.

Thus, in a further embodiment, the present invention also relates to storage stable aqueous compositions comprising water, Vitamin B12 and one or more preservatives selected from the group consisting of phenoxyethanol, (ethyl)hexylglycerin and 1,2-alkandiols, preferably from phenoxyethanol, ethylhexylglycerin and/or 1,2-hexandiol, wherein the composition further comprises psicose, optionally in the form of the sugar. It is well understood that all preferences and definitions given herein also apply. These compositions exhibit a particular storage stability in view of preventing/suppressing discoloration as well as storage stability overall.

The total amount of the preservatives selected from the group consisting of phenoxyethanol, (ethyl)hexylglycerin and 1,2-alkandiols, preferably from phenoxyethanol, ethylhexylglycerin and/or 1,2-hexandiol in the aqueous compositions according to the present invention is preferably selected from 0.1 to 5 wt.-%, more preferably 0.25 to 3 wt.-%, most preferably from 0.5 to 3 wt.-%, based on the total weight of the aqueous composition.

The topical cosmetic compositions according to the present invention may be leave-on or rinse-off compositions, and include any product applied to a human body, primarily for improving appearance, cleansing, odor control or general aesthetics. Preferably the cosmetic compositions of the present invention are leave-on compositions.

It is well understood that the topical cosmetic compositions according to the invention may next to water may comprise further ingredients as cosmetically acceptable carrier.

The term ‘cosmetically acceptable carrier’ (also referred to herein as carrier) refers to all vehicles/carriers conventionally used in cosmetic compositions, i.e. which are suitable for topical application to the keratinous tissue, have good aesthetic properties, are compatible with the actives present in the composition, and will not cause any unreasonable safety or toxicity concerns. Such carriers are well-known to one of ordinary skill in the art, and can include one or more compatible liquid(s) or solid filler diluent(s), excipient(s), additive(s) or vehicle(s) which are suitable for application to skin.

The exact amount of carrier will depend upon the actual level of the active ingredients and of any other optional ingredients that one of ordinary skill in the art would classify as distinct from the carrier (e.g., other active ingredients).

The compositions of the present invention preferably comprise from about 50% to about 99.999%, more preferably from about 60% to about 99.99%, still more preferably from 75% to about 99%, and most preferably, from about 80% to about 98% such as about 90% to about 98%, by weight of the composition, of a carrier, based on the total weight of the composition.

In a particular advantageous embodiment, the carrier consists furthermore of at least 30 wt. %, more preferably of at least 40 wt.-%, most preferably of at least 45 wt.-% of water, such as in particular of 50 to 90 wt.-% of water.

The cosmetic compositions in accordance with the invention can be in the form of a liquid, lotion, a thickened lotion, a gel, a cream, a milk, an ointment, a paste, a powder, a make-up, or a solid tube stick and can be optionally be packaged as an aerosol and can be provided in the form of a mousse such as an aerosol mousse, a foam or a spray foam, a spray, a stick.

Preferably the Vitamin B12 and the psicose are formulated into lotions, creams, gels, and tonics. These product forms may be used for a number of applications, including, but not limited to, hand and body lotions, facial moisturizers, anti-ageing preparations, make-ups including foundations, and the like. Any additional components required to formulate such products vary with product type and can be routinely chosen by one skilled in the art.

If the cosmetic compositions of the present invention are formulated as an aerosol and applied to the skin as a spray-on product, a propellant is added to the composition.

The cosmetic compositions according to the present invention can be prepared by conventional methods in the art such as e.g. by admixing the Vitamin B12 and psicose with all the definitions and preferences given herein with the cosmetically acceptable carrier.

The cosmetic composition may comprise further ingredients, which may form part of the carrier. Such ingredients are particularly surfactants, emulsifiers, thickeners, and oils. Such suitable surfactants, emulsifiers, thickeners, and oils are well known to a person skilled in the art.

The cosmetic compositions of the invention (including the carrier) may comprise further conventional (cosmetic) adjuvants and additives, such as preservatives/antioxidants, fatty substances/oils, water, organic solvents, silicones, thickeners, softeners, emulsifiers, antifoaming agents, aesthetic components such as fragrances, surfactants, fillers, anionic, cationic, non-ionic or amphoteric polymers or mixtures thereof, propellants, acidifying or basifying agents, dyes, colorings/colorants, abrasives, absorbents, chelating agents and/or sequestering agents, essential oils, skin sensates, astringents, pigments or any other ingredients usually formulated into such compositions.

If nothing else is stated, the excipients, additives, diluents, etc. mentioned in the following are suitable for the compositions according to the present invention. The necessary amounts of the cosmetic and dermatological adjuvants and additives can, based on the desired product, easily be determined by the skilled person.

The additional ingredients can either be added to the oily phase, the aqueous phase or separately as deemed appropriate. The mode of addition can easily be adapted by a person skilled in the art.

Examples of cosmetic excipients, diluents, adjuvants, additives as well as active ingredients commonly used in the skin care industry which are suitable for use in the cosmetic compositions of the present invention are for example described in the International Cosmetic Ingredient Dictionary & Handbook by Personal Care Product Council (http://www.personalcarecouncil.org/), accessible by the online INFO BASE (http://online.personalcarecouncil.org/jsp/Home.jsp), without being limited thereto.

The cosmetically active ingredients useful herein can in some instances provide more than one benefit or operate via more than one mode of action.

Of course, one skilled in this art will take care to select the above mentioned optional additional ingredients, adjuvants, diluents and additives and/or their amounts such that the advantageous properties intrinsically associated with the combination in accordance with the invention are not, or not substantially, detrimentally affected by the envisaged addition or additions.

The cosmetic compositions according to the present invention are in particular skin care preparations, functional preparations and/or hair care preparations such as most in particularly skin or hair care preparations.

Examples of skin care preparations are, in particular, light protective preparations (sun care preparations), anti-ageing preparations, preparations for the treatment of photo-ageing, body oils, body lotions, body gels, treatment creams, skin protection ointments, moisturizing preparations such as moisturizing gels or moisturizing sprays, face and/or body moisturizers, make-up as well as skin lightening preparations.

Examples of functional preparations are cosmetic compositions containing active ingredients such as hormone preparations, vitamin preparations, vegetable extract preparations, anti-ageing preparations, and/or antimicrobial (antibacterial or antifungal) preparations without being limited thereto.

Examples of hair care preparations which are suitable according to the invention and which may be mentioned are shampoos, hair conditioners (also referred to as hair rinses), hairdressing compositions, hair tonics, hair regenerating compositions, hair lotions, water wave lotions, hair sprays, hair creams, hair gels, hair oils, hair pomades or hair brilliantines. Accordingly, these are always preparations which are applied to the hair and the scalp for a shorter or longer time depending on the actual purpose for which they are used.

In a preferred embodiment, the cosmetic compositions according to the present invention are emulsions and/or gels. Even more preferably, the cosmetic compositions are emulsions which contain an oily phase and an aqueous phase such as in particular O/W, W/O, Si/W, W/Si, O/W/O, W/O/W multiple or a pickering emulsions.

The amount of the oily phase (i.e. the phase containing all oils and fats including the polar oils) present in such emulsions such as in particular O/W, W/O, Si/W, W/Si, O/W/O, W/O/W multiple or a pickering emulsions is preferably at least 10 wt.-%, such as in the range from 10 to 60 wt. %, preferably in the range from 15 to 50 wt.-%, most preferably in the range from 15 to 40 wt.-%, based on the total weight of the composition.

The oil phase according to the invention preferably comprises oils selected from butylenglykoldicaprylat/-dicaprat, propylenglykoldicaprylat/-dicaprat, dicaprylylether, C12-15-Alkylbenzoat, C18 38 fatty acid triglyceride, dibutyladipate, cyclomethicone, dimethicone, 2-phenylethylbenzoat, isopropyl lauroyl sarkosinate, caprylic/capric triglyceride as well as mixtures thereof.

The amount of the aqueous phase present in such emulsions is preferably at least 20 wt.-%, such as in the range from 20 to 90 wt.-%, preferably in the range from 30 to 80 wt.-%, most preferably in the range from 30 to 70 wt.-%, based on the total weight of the composition.

Advantageously in all emulsions of the present invention the ratio of oily phase to aqueous phase is selected in the range from 40:60 to 30 to 70.

In one particular advantageous embodiment, the compositions according to the present invention are in the form of an oil-in-water (O/W) emulsion comprising an oily phase dispersed in an aqueous phase in the presence of an O/W emulsifier. The preparation of such O/W emulsions is well known to a person skilled in the art.

If the composition according to the invention is an O/W emulsion, then it contains advantageously at least one O/W- or Si/W-emulsifier selected from the list of, glyceryl stearate citrate, glyceryl stearate SE (self-emulsifying), stearic acid, salts of stearic acid, polyglyceryl-3-methylglycosedistearate. Further suitable emulsifiers are phosphate esters and the salts thereof such as cetyl phosphate (e.g. as Amphisol® A from DSM Nutritional Products Ltd.), diethanolamine cetyl phosphate (e.g. as Amphisol® DEA from DSM Nutritional Products Ltd.), potassium cetyl phosphate (e.g. as Amphisol® K from DSM Nutritional Products Ltd.), sodium cetearylsulfate, sodium glyceryl oleate phosphate, hydrogenated vegetable glycerides phosphate and mixtures thereof. Further suitable emulsifiers are sorbitan oleate, sorbitan sesquioleate, sorbitan isostearate, sorbitan trioleate, cetearyl glucoside, lauryl glucoside, decyl glucoside, sodium stearoyl glutamate, sucrose polystearate and hydrated polyisobutene. Furthermore, one or more synthetic polymers may be used as an emulsifier. For example, PVP eicosene copolymer, acrylates/C10-30 alkyl acrylate crosspolymer, and mixtures thereof.

The at least one O/W, respectively Si/W emulsifier is preferably used in an amount of 0.5 to 10 wt. %, in particular in the range from 0.5 to 6 wt.-%, such as more in particular in the range from 0.5 to 5 wt.-%, such as most in particular in the range from 1 to 4 wt.-%, based on the total weight of the cosmetic composition.

Particular suitable O/W emulsifiers to be used in the compositions according to the invention encompass phosphate ester emulsifiers such as advantageously 8-10 alkyl ethyl phosphate, C9-15 alkyl phosphate, ceteareth-2 phosphate, ceteareth-5 phosphate, ceteth-8 phosphate, ceteth-10 phosphate, cetyl phosphate, C6-10 pareth-4 phosphate, C12-15 pareth-2 phosphate, C12-15 pareth-3 phosphate, DEA-ceteareth-2 phosphate, DEA-cetyl phosphate, DEA-oleth-3 phosphate, potassium cetyl phosphate, deceth-4 phosphate, deceth-6 phosphate and trilaureth-4 phosphate.

A particular suitable O/W emulsifier to be used in the compositions according to the invention is potassium cetyl phosphate e.g. commercially available as Amphisol® K at DSM Nutritional Products Ltd Kaiseraugst.

Another particular suitable class of O/W emulsifiers are non-ionic self-emulsifying systems derived from olive oil e.g. known as (INCI Name) cetearyl olivate and sorbitan olivate (chemical composition: sorbitan ester and cetearyl ester of olive oil fatty acids) sold under the tradename OLIVEM 1000.

In one particular embodiment, the invention relates to cosmetic compositions with all the definitions and preferences given herein in the form of O/W emulsions comprising an oily phase dispersed in an aqueous phase in the presence of an O/W emulsifier wherein the O/W emulsifier is potassium cetyl phosphate. The amount of oily phase in such O/W emulsions is preferably at least 10 wt.-%, more preferably in the range from 10 to 60 wt.-%, most preferably in the range from 15 to 50 wt.-%, such as in the range from 15 to 40 wt.-%, based on the total weight of the composition.

Preferably, the cosmetic compositions according to the invention further comprise at least one fatty alcohol (co-emulsifier), such as in particular cetyl alcohol, cetearyl alcohol and/or behenyl alcohol. The total amount of one or several fatty alcohols on the topical compositions according to the invention is preferably selected in the range from about 0.1 to 10.0 wt.-%, in particular in the range from about 0.5 to 6.0 wt.-% with respect to the total weight of the topical composition.

Preferably, the topical compositions according to the invention comprise a thickener in particular if the topical composition is in the form of an emulsion to assist in making the consistency of a product suitable. Preferred thickeners are aluminiumsilicates, xanthan gum, hydroxypropylmethylcellulose, hydroxyethylcellulose, polyacrylates such as Carbopole® (e.g. Carbopole 980, 981, 1382, 2984, 5984) or mixtures thereof. Further preferred thickeners encompass acrylate/C10-30 alkyl acrylate copolymers (such as e.g. Pemulen TR 1, Pemulen TR 2, Carbopol 1328 by. NOVEON) as well as Aristoflex AVC (INCI: Ammonium Acryloyldimethyltaurate/VP Copolymer).

The cosmetic compositions according to the present invention advantageously comprise one or more preservatives preservative. When present, the preservative is preferably used in an amount of 0.1 to 2 wt.-%, more preferably in an amount of 0.5 to 1.5 wt.-%, based on the total weight of the composition.

The cosmetic compositions according to the invention in general have a pH in the range from 3 to 10, preferably a pH in the range from 4 to 8 and most preferably a pH in the range from 4 to 7.5 such as in the range from 5 to 6.5. The pH can easily be adjusted as desired with suitable acids, such as e.g. citric acid, or bases, such as sodium hydroxide (e.g. as aqueous solution), triethanolamine (TEA Care), Tromethamine (Trizma Base) and Aminomethyl Propanol (AMP-Ultra PC 2000), according to standard methods in the art.

The amount of the cosmetic composition to be applied to the skin is not critical and can easily be adjusted by a person skilled in the art. Preferably the amount is selected in the range from 0.1 to 3 mg/cm2 skin, such as preferably in the range from 0.1 to 2 mg/cm2 skin and most preferably in the range from 0.5 to 2 mg/cm2 skin.

The following examples are provided to further illustrate the compositions and effects of the present invention. These examples are illustrative only and are not intended to limit the scope of the invention in any way.

### EXAMPLES

The formulations as outlined in table 1 and 2 were prepared and then stored in clear glass vials at 50° C. (accelerated stability test conditions) either for 2 weeks or for 3 months. The colour stability of Vitamin B12 was assessed by measuring the a value (L*a*b* values/CIELAB system) after the times as indicated in the tables (the lower the a-value, the higher the discoloration, i.e. fading of the red-pinkish color of Vitamin B12). In addition for the series depicted in table 2, the ΔE vs to was calculated (also here, the higher the value, the higher the discoloration).

TABLE 1

| TRADENAME | INCI | Control | Inv-1 | Ref-1 |
|---|---|---|---|---|
| WATER DEM. | Aqua | Ad 100 | Ad 100 | Ad 100 |
| Euxyl ® PE 9010 | Phenoxyethanol, | 1 | 1 | 1 |
| | Ethylhexylglycerin | | | |
| Vitamin B12 | Cyanocobalamine | 0.01 | 0.01 | 0.01 |
| Cryst Food Grade | | | | |
| Psicose | | — | 0.25 | 0.001 |
| a-value | t = 0 | 9.44 | 10.91 | 12.1 |
| | t = 2 weeks | 6.21 | 7.5 | 5.94 |
| Δ a [%] | | −34.2 | −31.3 | −50.9 |

TABLE 2

| | | | Invention | | Reference | |
|---|---|---|---|---|---|---|
| TRADENAME | INCI | Control | 1 | 2 | 1 | 2 |
| WATER DEM. | Aqua | Ad 100 | Ad 100 | Ad 100 | Ad 100 | Ad 100 |
| Euxyl ® PE 9010 | Phenoxyethanol, | 1 | 1 | 1 | 1 | 1 |
| | Ethylhexylglycerin | | | | | |
| Vitamin B12 | Cyanocobalamine | 0.01 | 0.01 | 0.01 | 0.01 | 0.01 |
| Cryst Food | | | | | | |
| Grade | | | | | | |
| Psicose | | — | 0.25 | 0.001 | — | — |
| D-Sorbitol | Sorbitol | — | — | — | 0.001 | 0.25 |
| a-value | t = 3 months | 2.95 | 7.77 | 2.96 | 3.09 | 4.44 |

1. Use of psicose for suppressing discoloration in aqueous compositions comprising Vitamin B12 and a preservative selected from the group consisting of phenoxyethanol and (ethyl)hexylglycerin, wherein the amount of psicose is selected in the range from 0.005 to 0.5 wt.-% and the amount of water is at least 30 wt. %, based on the total weight of the aqueous composition.

2. The use according to claim 1, wherein the amount of psicose in the aqueous composition is selected in the range from 0.01 to 0.5 wt.-%, preferably from 0.1 to 0.5 wt.-%, based on the total weight of the aqueous composition.

3. The use according to claim 1, wherein the amount of Vitamin B12 in the aqueous composition is selected in the range from 0.0001 wt.-% to 0.1 wt.-%, preferably from 0.001 wt.-% to 0.05 wt.-%, most preferably from 0.001 to 0.025 wt.-%, based on the total weight of the aqueous composition.

4. The use according to claim 1, wherein the psicose is D-psicose.

5. The use according to claim 1, wherein the aqueous composition n further comprises both of phenoxyethanol and (ethyl)hexylglycerin, preferably phenoxyethanol and ethylhexylglycerin.

6. The use according to claim 1, wherein the total amount of the preservative in the aqueous composition is selected in the range from 0.1 to 5 wt.-%, more preferably 0.25 to 3 wt.-%, most preferably from 0.5 to 3 wt.-%, based on the total weight of the aqueous composition.

7. The use according to claim 1, wherein the amount of water in the aqueous composition is at least 40 wt.-%, preferably at least 45 wt.-%, such as in particular in the range from 50 to 99 wt.-%, based on the total weight of the aqueous composition.

8. A method for reducing discoloration of an aqueous composition containing Vitamin B12 and a preservative selected from the group consisting of phenoxyethanol and (ethyl)hexylglycerin, said method comprising preparing an aqueous composition by admixing Vitamin B12, phenoxyethanol and/or (ethyl)hexylglycerin, psicose and water, wherein the amount of psicose is selected in the range from 0.005 to 0.5 wt.-% and the amount of water is at least 30 wt. %, based on the total weight of the aqueous composition and storing said aqueous composition for at least one week, preferably for at least 2 weeks, such as most preferably for at least 3 months.

9. The method according to claim 8, wherein the psicose is D-psicose.

10. The method according to claim 8, wherein the aqueous composition further comprises both of phenoxyethanol and (ethyl)hexylglycerin, preferably phenoxyethanol and ethylhexylglycerin.

11. An aqueous composition comprising water, Vitamin B12, a preservative selected from the group consisting of phenoxyethanol and (ethyl)hexylglycerin, and psicose, wherein the amount of psicose is selected in the range from 0.005 to 0.5 wt.-% and the amount of water is at least 30 wt.-%, based on the total weight of the aqueous composition.

12. An aqueous composition according to claim 11 wherein the psicose is D-psicose.

13. An aqueous composition according to claim 11, wherein the composition comprises both of phenoxyethanol and (ethyl)hexylglycerin, preferably phenoxyethanol and ethylhexylglycerin.
